(19) United States
(12) Patent Application Publication
(10) Pub. No.: US 2026/0215711 A1
(43) Pub. Date: Jul. 30, 2026

(54) WEARABLE BIOSENSOR DEVICE WITH IMPROVED COVER AND IMPROVED FLEXIBILITY AND CONFORMANCE

(71) Applicant: AQUILX INC., Bonsall, CA (US)

(72) Inventors: Farshad TEHRANI, San Diego, CA (US); Hazhir TEYMOURIAN, San Diego, CA (US); Brian WUERSTLE, Pacifica, CA (US)

(21) Appl. No.: 19/145,786

(22) PCT Filed: Jan. 8, 2024
(86) PCT No.: PCT/US2024/010775

§ 371 Date: Jul. 3, 2025

Related U.S. Application Data
(60) Provisional application No. 63/437,445, filed on Jan. 6, 2023.
(60) Provisional application No. 63/437,583, filed on Jan. 6, 2023.

(51) Int. Cl. A61B 5/1473; A61B 5/00; A61B 5/145; A61M 37/00

(52) U.S. Cl. CPC A61B 5/14735; A61B 5/14514; A61B 5/14546; A61B 5/4266; A61B 5/6833; A61B 5/685; A61B 5/7225; A61B 5/7271; A61M 37/00; A61B 2562/0247; A61B 2562/0261; A61B 2562/0271; A61B 2562/028; A61B 2562/066; A61B 2562/164; A61B 2562/227; A61M 2202/0007; A61M 2202/04; A61M 2210/04

(57) ABSTRACT

A wearable biosensor device with an improved cover to improve placement of the microneedles and to secure them during the life of the sensor. Improved flexibility and conformability of the device also aids in placing and securing.

### STATEMENT REGARDING RELATED APPLICATIONS

This application claims priority to, and the full benefit of, U.S. provisional patent application No. 63/437,445 filed Jan. 6, 2023 and U.S. provisional patent application 63/437,583 filed Jan. 6, 2023, and both applications are incorporated herein.

This application incorporates by reference PCT/US22/36424 and U.S. Pat. No. 11,684,298 issued Jun. 17, 2023 (together, “the '298 patent”), as if set forth fully herein.

### ASPECTS OF THE INVENTION

The present invention is an improvement over the invention in the '298 patent. As with the '298 patent include that this invention also comprises a construction of the microneedle array comprising a substrate integral with microneedles cast of nonconductive polymers in a mold coated with an electrically conductive layer and then overlaid with at least one chemical layer functionalized for reactions with target analytes in a biofluid such as interstitial fluid, blood or cerebrospinal fluid. The present invention is a wearable biosensor device system overlapping with the '298 patent but improved for flexibility and conformance to the skin of a wearer. As discussed herein, these improvements include, without limitation, a better and extended cover for the microneedle array, different electrical interconnections and additional means for flexibility of the device and conformance to the user's skin thus improving insertion and securing the microneedles in place.

The improvements herein include embodiments which are fully disposable and others which are partially disposable and partially reusable. In all embodiments, the device is designed to provide robust mating between a biosensor device and a user's skin at the time of insertion and over the life of the biosensor. The device includes the improvement of seating the microneedle array in a lower surface of an extended cover, the lower surface of the extended cover surrounded by an upper surface. so that the base of each of the microneedles is recessed below a fillet at the inner boundary of the outer surface so that the fillet is configured to pinch a user's skin where the microneedles are inserted into the skin. By mitigating dynamic interference sources (e.g., device movement when the user is moving vigorously) and static/quasi-static interference sources (e.g., the user sleeps on the device). These aspects enable a) dynamic and static artifact mitigation or suppression, b) high accuracy bio-molecular sensing, and c) safe ultra-smooth microneedle insertion.

Some of the improvements described herein apply to wearable biosensor devices of several kinds. One kind of device is the polymer-based construction of a nonconductive polymer microneedle array coated with an electrically conductive layer and sealed and strengthened by a nonconductive polymer cover unit, a version of which is described in the '298 patent, incorporated herein. Another kind of wearable biosensor device comprises a microneedle array at least partially formed from a semiconductor (e.g., silicon) substrate and includes various material layers applied and shaped using various suitable microelectromechanical systems (MEMS) manufacturing techniques (e.g., deposition and etching techniques) known in the art. Suitable configurations for the MEMS device described herein are found in US patent publication number US20220031209A1, which is also incorporated herein.

### EXAMPLES OF THE INVENTION

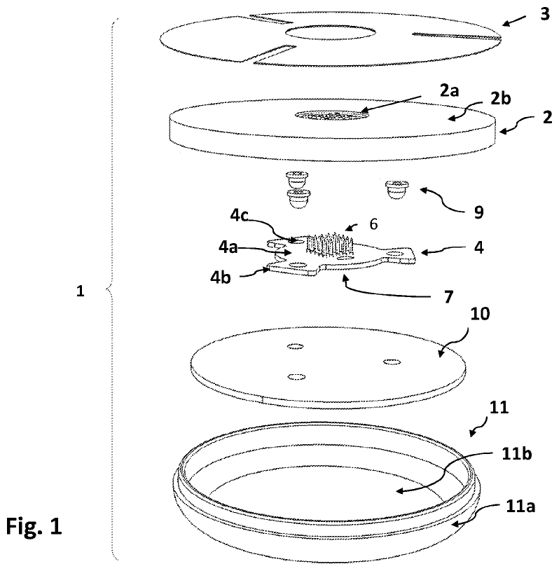

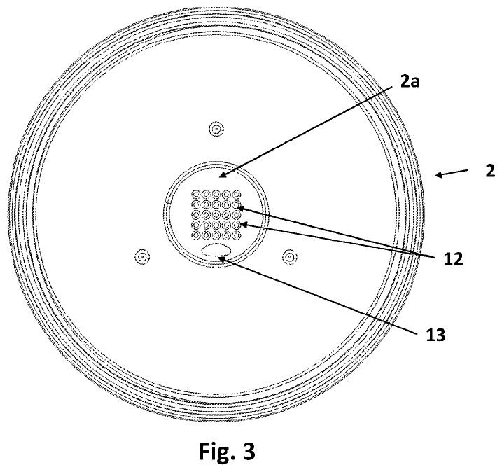

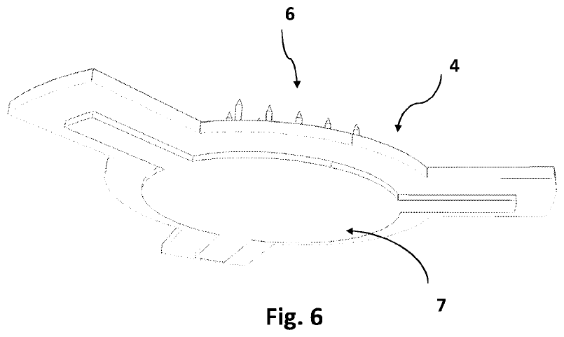

FIG. 1 is an exploded perspective view of one embodiment of the improved wearable biosensor device 1. This embodiment is intended to be replaceable at the end of the life of the device, that is, it is a single unit which is fully disposable. An extended cover 2 comprises a lower surface 2a, here in the center (without sleeves), surrounded by an upper surface 2b. At a fillet 2c, a rounded annular junction at the inner boundary of the upper surface, a downward slope 2d of the extended cover descends to the lower surface. The extended cover also has at least one sidewall 2e which secures and seals it in complementary fashion to a sidewall 11a of the lower enclosure 11 which has a bottom 11b. The lower surface of the extended cover has a set of first openings 12 (holes) which are complementary to, and correspond with, microneedles 6 of the microneedle array 4. The first openings can be seen clearly in FIG. 3. A flexible microneedle array 4 comprises in its center a substrate 4a and microneedles 6 which are integral with one another. In this embodiment, a force touch sensor 7 underneath the microneedle array is shown, for example, in FIG. 6. An electronics unit 10 here is a printed circuit board (PCB). Conductive pins 9a on the cantilevered arms 4b of the microneedle array at to the electronics unit. Connection holes 4c allow a connection to contact points 10a on the electronics unit by conductive pins 9a which may be flexible and elastic or rigid. A skin adhesive layer 3 is attached to the upper surface of the extended cover, that is, there is an opening in the skin adhesive layer 3 above the microneedles as they would appear through the first openings in the lower surface. The skin adhesive layer may have slots 3a to prevent wrinkling.

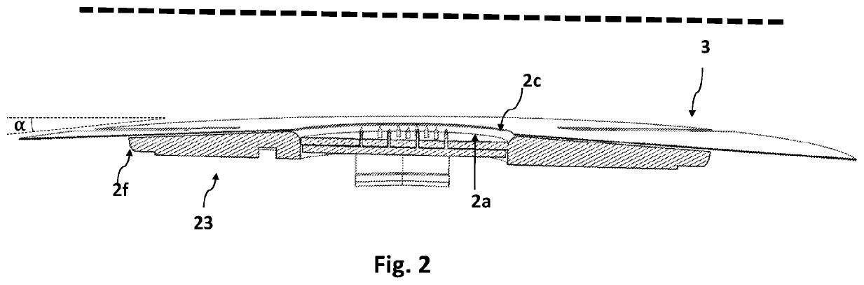

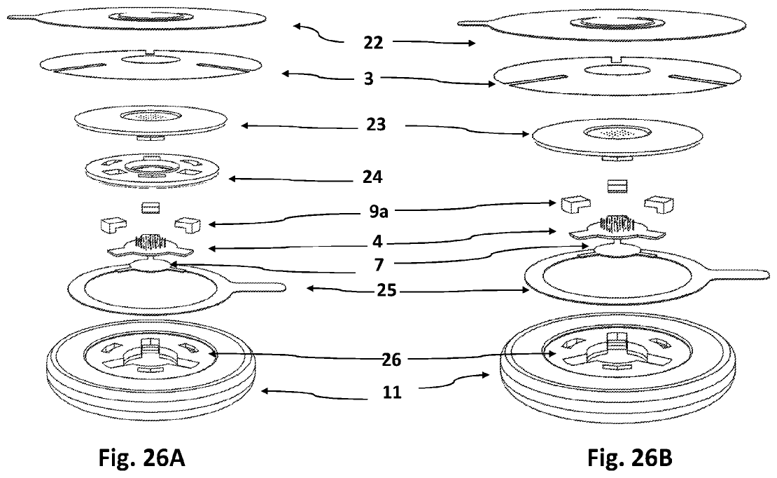

FIG. 2 is a section view of the extended cover 2 as used in a replaceable module 18a (shown for example in FIG. 26A) with the skin adhesive layer 3 attached tightly. This embodiment of the extended cover has no sidewall as does the disposable device in FIG. 1. An angle alpha α shows the outer portion 2f of the upper surface 2b is lower than the inner boundary at the fillet 2c.

FIG. 3 is the under side of the extended cover 2 showing the first openings 12 in the lower surface 2a. An optional second opening 13 allows the flow of curable custom resin in manufacturing. There are PILLARs to hold the elastic conductive pegs in place. The elastic conductive pegs (FIG. 1, feature 9) pull on the pillars from their side with hole. They are to align the microneedle array at the right place on the extended cover 2.

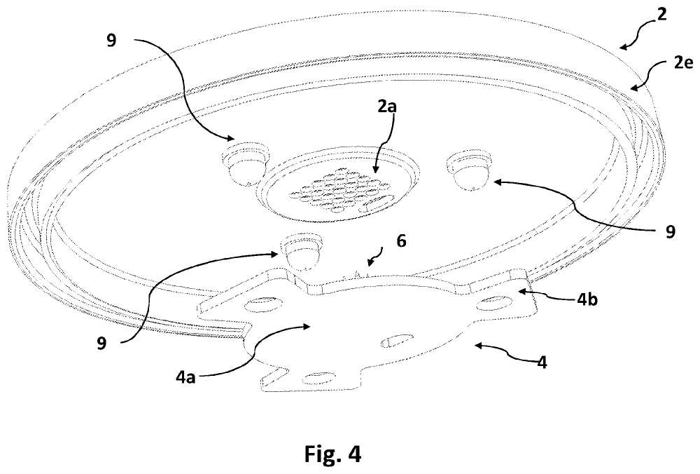

FIG. 4 is a perspective view of the under side of the extended cover 2 with the lower surface 2a and the first openings 12. The microneedle array 4 is positioned for the microneedles 6 to be inserted through the first openings. The cantilevered arms 4b having connection holes 4c surround the substrate 4a in the center of the microneedle array.

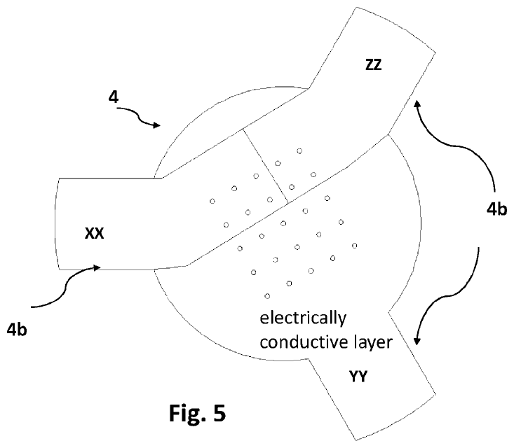

FIG. 5 is a top down view of the microneedle array 4 showing the microneedles 6 in the center substrate 4a. All electrode regions 14, here three, are electrically separated from one another. Each region comprised at least one microneedle. Each electrode region functions as at a working electrode, counter electrode or a reference electrode. At least two electrode regions comprising a working electrode and a combined counter-reference electrode is constructed to form a complete electrochemical system. FIG. 5 displays an example of a three-electrode electrochemical sensing system comprising a working electrode XX (six microneedles), a counter electrode YY (15 microneedles), and a reference electrode (4 microneedles) ZZ. Each electrode region has an electrically conductive layer 15 connected to one conductive pin 9 at a connection hole 4c.

FIG. 6 is a perspective view from underneath the microneedle array 4 showing the cantilevered arms 4b and a force touch sensor 7, here a strain gauge. The tips 6a of microneedles, and the body regions 6b, are shown.

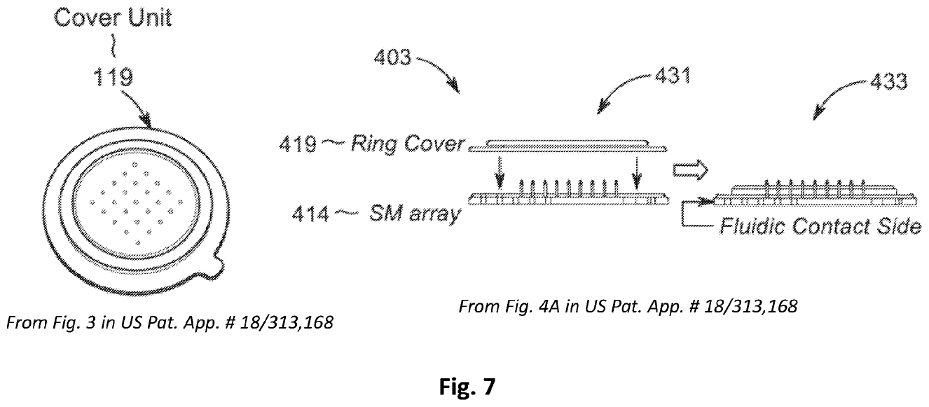

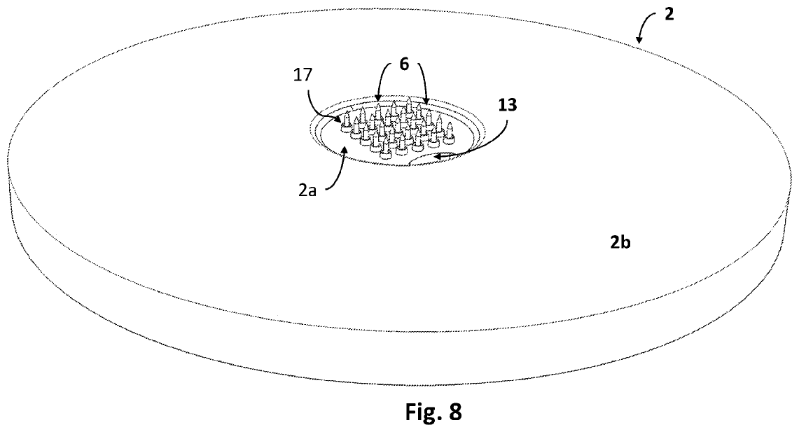

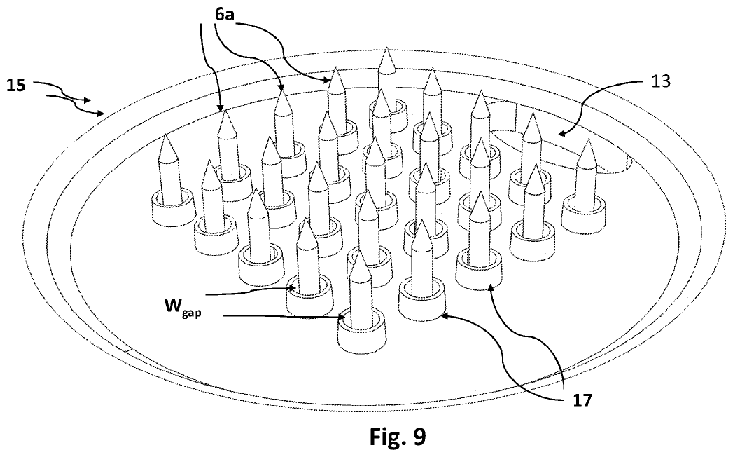

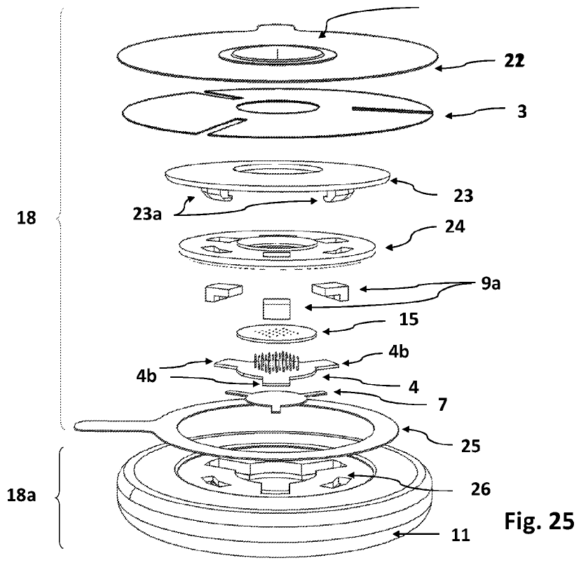

The present invention includes at least two embodiments of an improved cover over the '298 patent. The two embodiments are the extended cover 2 and a separate cover 15, and each of these comprises improvements not found in the '298. The '298 patent incorporates a basic cover, for example in FIGS. 3 and 4A in the '298 patent, collected here in FIG. 7 for convenient reference. The basic cover is flat as shown in features numbered 119 and 419. The present invention, however, is a novel improvement over the device with the basic cover. The improvements herein include one or more sleeves 17 extending upward around the first openings 12 through which the microneedles of the microneedle array are pressed. Another improvement are bodily features 20 which can perform different functions as described further herein. Another improvement are conductive traces 19 which have important functions also discussed herein. These improvements may be used separately or in tandem. In FIG. 8 sleeves 17 are shown on the lower surface 2a of the extended cover 2. Microneedles 6 extend above the sleeves. In this embodiment, the sleeves are uniform but they may be varied in shape and size, or may be present only for a portion of the first openings, in various embodiments. An optional second opening 13 is also shown. FIG. 9 shows the embodiment of the separate cover 15 with sleeves 17 for use in the replaceable module 18a, as shown in FIG. 25. Gaps W_{gap} between the sleeves 17 and the microneedles 6 are shown here.

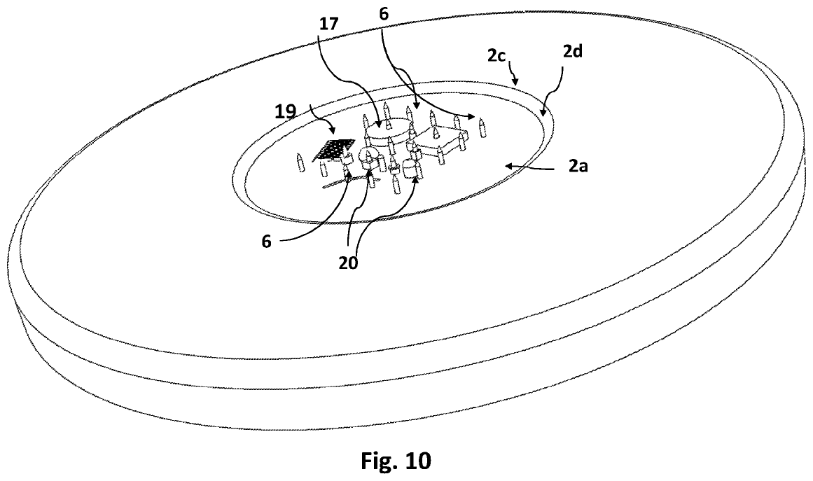

FIG. 10 is a perspective view of an embodiment of the extended cover 2 with sleeves 19 and bodily features 20 on the lower surface 2a. The fillet 2c is at the inner boundary of the upper surface 2b where the slope 2d begins to descend to the lower surface 2a. The microneedles of the microneedle array have been inserted through the first openings 12, some with sleeves and some without sleeves. A conductive trace 19 is to the side of the sleeves 19 and microneedles 6. This embodiment has a sidewall 2e.

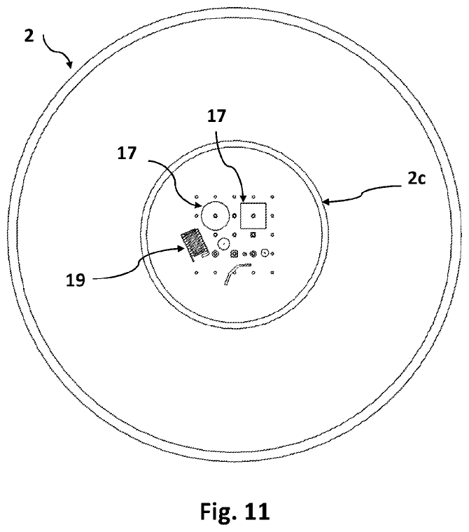

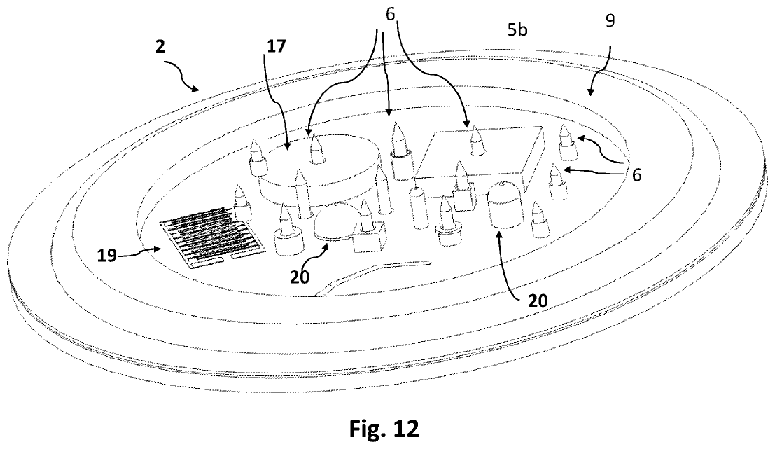

FIG. 11 is a top down view of an extended cover similar to that shown in FIG. 10, showing different sized and shaped sleeves 17 and bodily features 20. The fillet 2c, slope 2d, first openings 12, conductive trace 19 and optional second opening 13 are also shown. FIG. 12 is another perspective view of the extended cover 2 with the microneedle array inserted on the under side of the extended cover, so that the microneedles 6 extend beyond sleeves. Bodily features 20 are interspersed. The fillet 2c, slope 2d, conductive trace 19 are also shown.

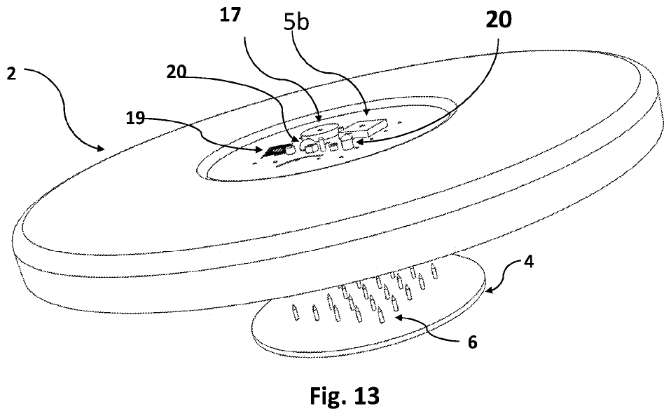

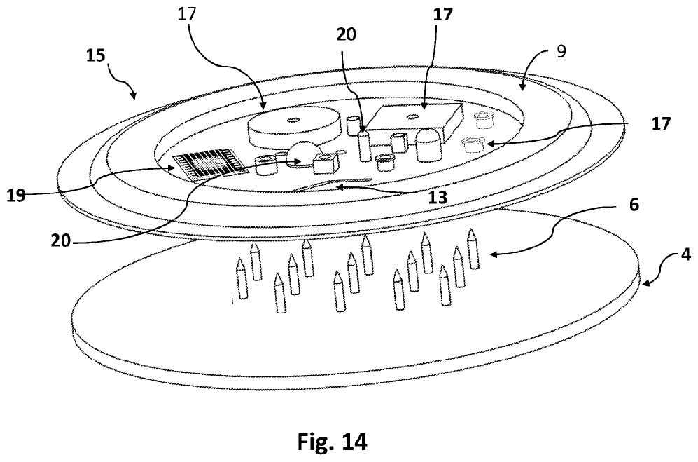

FIG. 13 is another perspective view of the extended cover 2 with a microneedle array 4 without cantilevered arms in position for insertion into the first openings, some with sleeves and some without. FIG. 14 is a perspective view of the improved separate cover 15 having features to similar to those in FIG. 13.

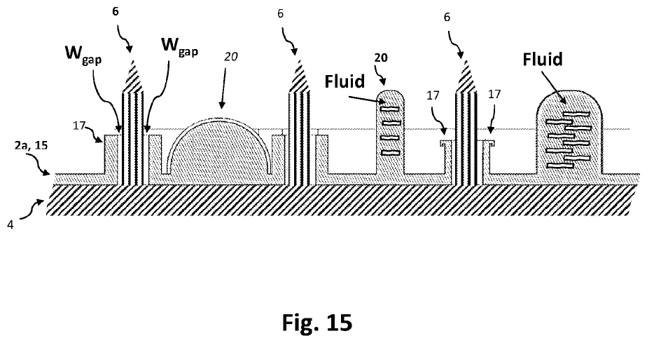

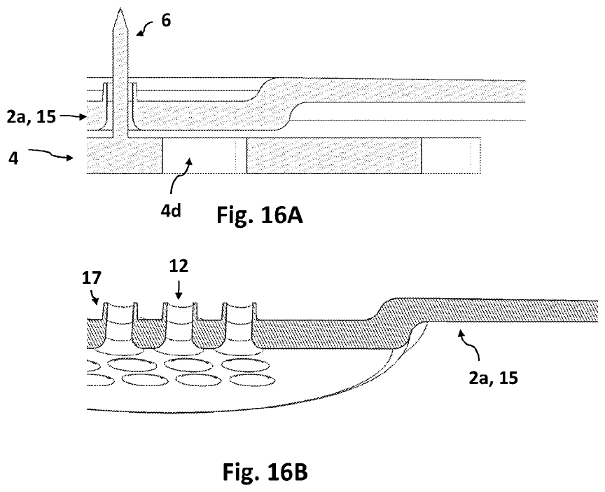

Cross sections show additional detail. FIG. 15 is a side section view of a portion of the cover having improvements (either 2 or 15). The gap between the outer diameter of the microneedles 6 and the inner diameter of the sleeves is Wgap. Bodily features 20 are shown holding fluid. The sleeve furthest to the right has arms which extend outward from the sleeve. FIG. 16A is a section side view of one microneedle 6 extending through a first opening before cured custom resin is flowed into W_{gap} through, for instance, an opening 4d in the microneedle array 4. Here, there is a space 21 configured to be between the cover 2, 15 and microneedle array 4 so that curable resin can flow through and secure the cover to the microneedle array, and also to seal the W_{gap}. The curable resin, once cured, provides strength and protection of the underlying electronics from fluids.

FIG. 16B is a section perspective view of a portion of the under side of the cover 2, 15 showing the first openings 12 surrounded by sleeves 17.

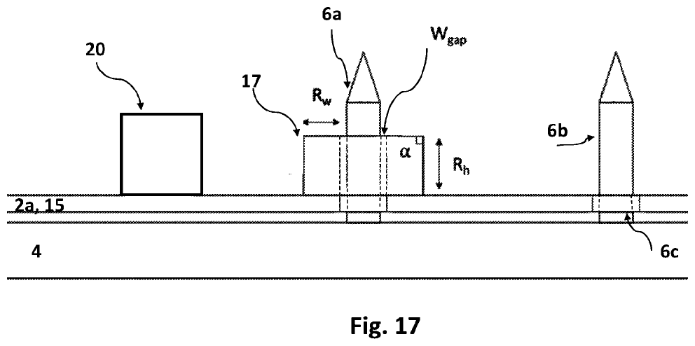

FIG. 17 is a side view of a portion of the cover 2, 15 with a sleeve 17 surrounding a portion of the microneedle body 6b and the base 6c and leaving a portion of the body and the tip 6a exposed for insertion into the user's skin. One of the microneedles has no sleeve. A bodily feature 20 is also shown. In the improved cover shown e.g. in FIG. 17, there is a wide range of features and functions that stem from different combinations of variables of the radius width (R_{w}), the width of the gap between the sleeve and the microneedle (W_{gap}), and the height of the radius (Rh), and the angle α. For example, the angle α in FIG. 17 is a right angle, but if it were increased to 135 degrees on the entire annulus, it would contribute to easier insertion. The properties of the materials of the sleeves and improved cover itself that can be distinct from the materials used in the microneedle array all of which are described herein. A bodily feature 20 can be either mechanical or chemical in nature. When mechanical, it deforms the skin by stretching or pinching the skin near one or more microneedles in order to make the microneedles pierce the skin more easily. When chemical, iontophoresis can be used to improve conductance of compounds loaded in the bodily feature, and the compounds can cross the skin more easily than without application of the voltage. The compounds can be in solution, gels or semi-solids. Also, bodily features can be chemical and mechanical at the same time. Reproducibility of sealing the microneedles is enabled in the improved cover with sleeves if further enhanced by adjusting the height of the sleeve to fine tuning of the available electrochemical surface area on the microneedles by mainly adjusting the height (Rh) among all sleeves. The microfluidic channel (adjustable by Wgap) between the interior wall of the sleeve and the microneedle creates a microfluidic pathway for the insulating resin which covers the base of the microneedle until it reaches the top of the sleeve. The micromachining method (CNC) used to fabricate the covers allows highly precise (~500 nm) control/reproducibility over the sleeves' heights and therefore, and there is a high level of control over the surface areas of the microneedles. The sleeve heights may be selected to be to be uniquely adjustable for each and every microneedle and/or microneedle group (e.g., working electrodes, vs. counter electrodes, vs. reference electrodes). The adjustability of the sleeves allows precision skin penetration, that is, tuning among different humans with different skin thickness etc. Adjusting the sleeve height controls the height of the microneedle which penetrates the skin. When microneedles pierce the outermost layer of the skin, the intersection of the microneedle and the sleeve act as a stopping point due to the bigger diameter. This provides leverage to control the skin penetration depth of the microneedles depending on the specific skin anatomical properties (e.g., different inter-individual skin thicknesses).

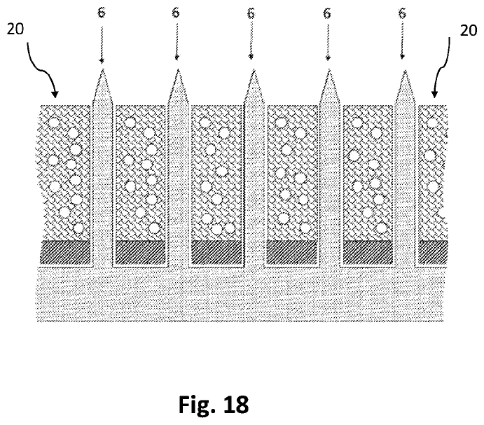

FIG. 18 is a section view of a microneedle array 4 and a cover 2, 15 with spongy, elastic and absorbable bodily features 6 impregnated with fluid containing compounds selected from the group consisting of pharmaceuticals, salts, anti-inflammatory compounds and the like. These compounds can be intended for absorption by the skin or for combatting biofouling of the microneedles to extend their lives.

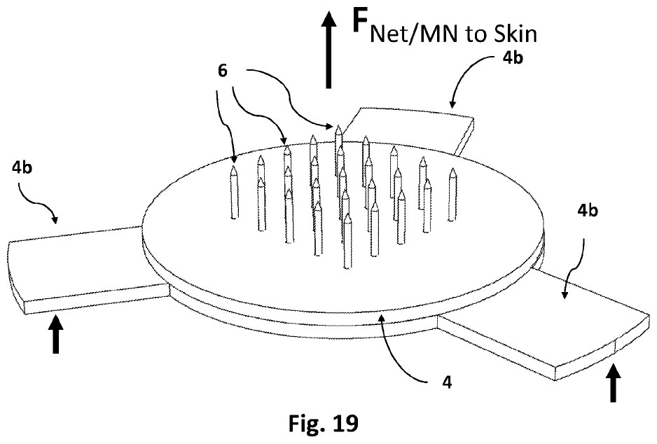

FIG. 19 is a perspective view of the microneedle array 4 with cantilevered arms 4b. Arrows show F_{net/Microneedle to skin} which is the amount of net force applied from the microneedles to the skin during the microneedle insertion at the time of sensor application to the wearer's body and also during the sensor wear period.

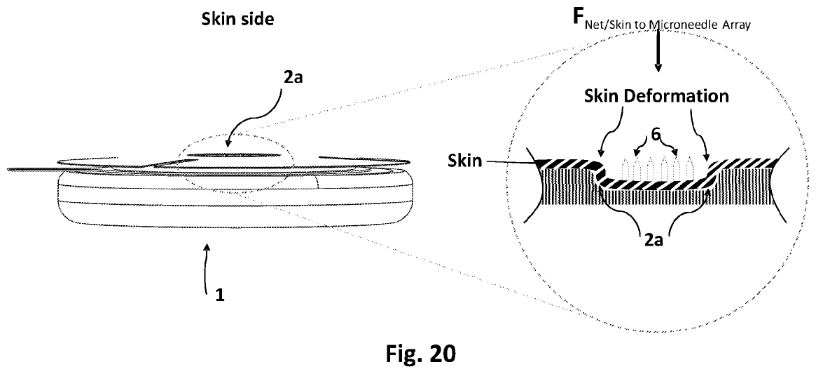

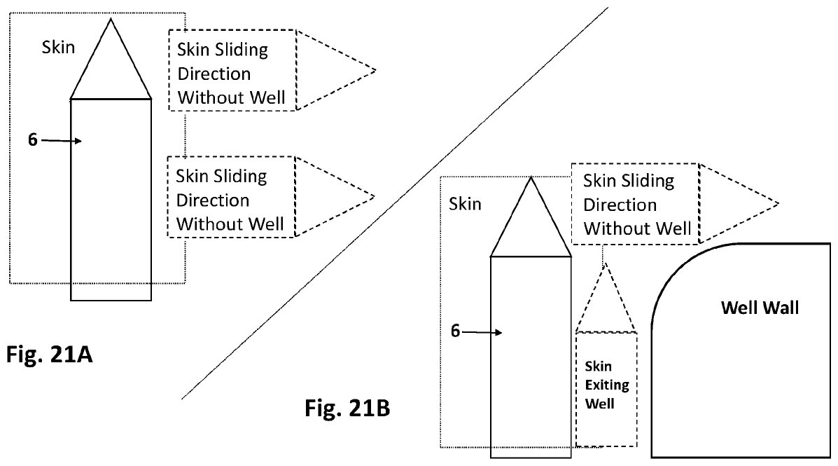

As shown in FIG. 20, the microneedle array recessed below the upper surface of the cover, which causes the deformation of the skin at the positive fillet 2c and the downward slope 2d (negative fillet) which, due to the inherence elasticity of the skin, provides constant and passive microneedle pressure against the skin while the wearable device is adhered to skin. That is, when the area surrounding the microneedle array is depressed more into the skin, such as with a ring-protrusion surrounding the microneedle. This constant passive pressure at the raised areas causes the skin to stretch into the recess below the fillet, thereby immobilizing the skin from moving from normal-to-microneedle-tip insertion of the microneedle to the skin. Said differently, the well prepares the skin for a smooth microneedle insertion by 1) stretching the skin, 2) immobilizing the skin from the sides during microneedle insertion, 3) causing a constant compression force from the skin to the microneedle due to the constant pinching of the elastic skin induced by the fillet 2c, and 4) further mechanical protection of the microneedles at all microneedle/skin interaction including the initial application of the sensor on body and microneedle/skin insertion, during wear, final removal, and incidental sliding of the microneedle on body primarily by shifting the fracture inducing normal stress from the lower body of the microneedle, in the lack of a well, to the rather bending inducing normal stress to the body to the upper body of the microneedles. This is shown in FIG. 20 and FIGS. 21A and 21B. The microneedle array illustrated from top view, left, and side view, right, with drawing of the skin formation after application of the device to the skin. FIG. 21A depicts the skin sliding sideways without a well wall but FIG. 21A shows the skin is pinched and rises to the top of the well wall. Pinching force shown in FIG. 21A where the skin sliding stress is applied through the entire body of the microneedle array and FIG. 21B with well where the skin skidding force induces the “skin-exiting-well” stress to shift the forces away from the bottom of the microneedle array to the surface while skin is being exited out of the well.

FIG. 20 is a side view of the wearable biosensor device 1 of the embodiment with two modules 18, 18a and an enlarged section view of the user's skin being deformed where the microneedles 6 have pierced the skin in the area defined by the fillet 2c and the downward slope to the lower surface 2a of the extended cover or the separate cover 15. FIG. 21A is a schematic showing the sideways movement of the skin when being pierced by a microneedle and FIG. 21B shows how the movement of the skin is deflected upwards by the slope.

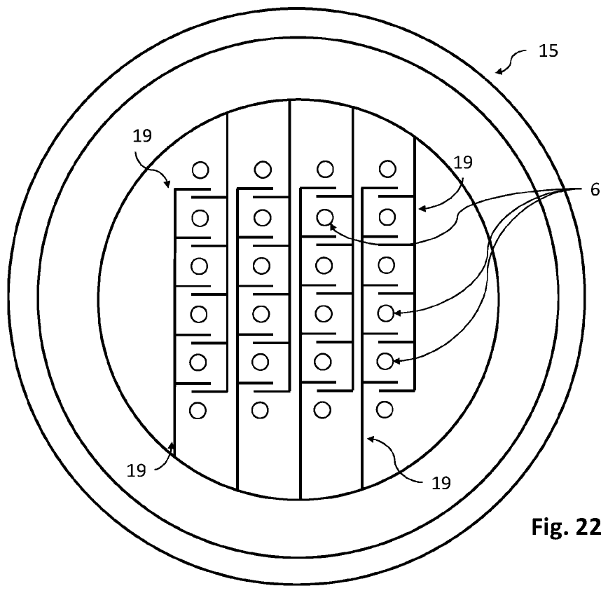

FIG. 22 is a top down view of a separate cover 15 or the lower surface 2a of an extended cover in which the first openings are dispersed among the conductive trace 20.

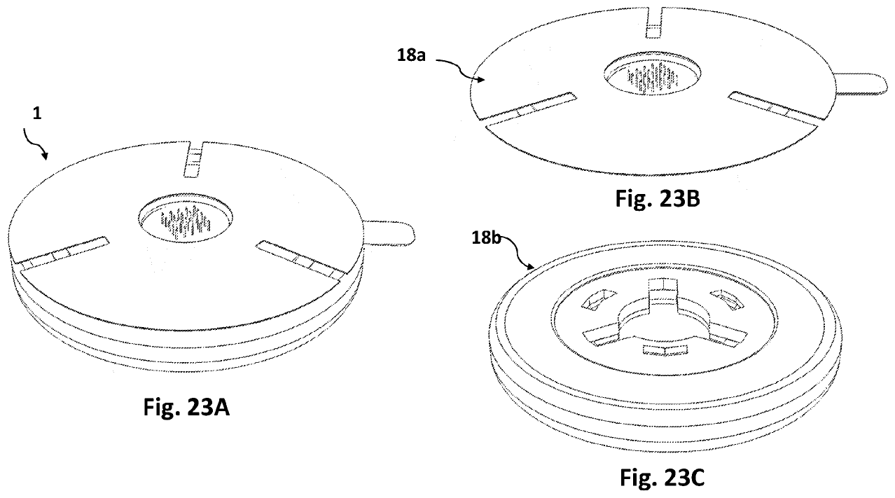

FIG. 23A is a perspective view of one embodiment of the wearable biosensor device 1 as a two module device, with the replaceable module 18a in FIG. 23B and the reusable module 18b in FIG. 23C.

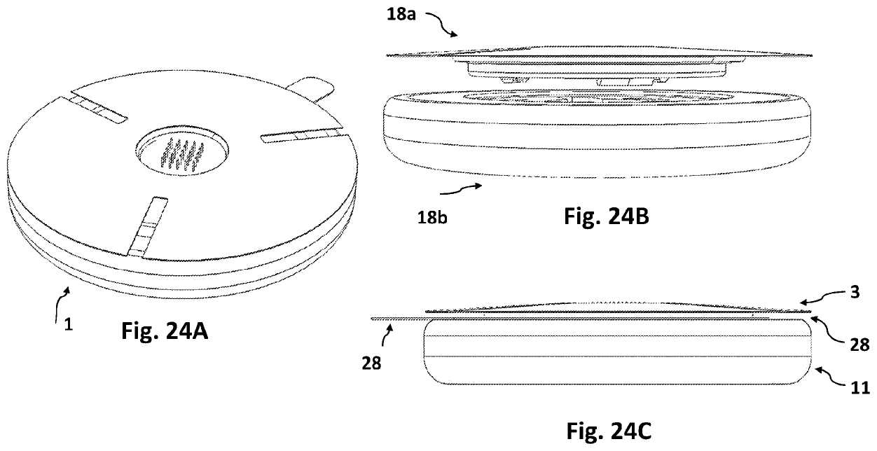

FIG. 24A is similar to FIG. 23A, with FIG. 24B showing the two modules 18a, 18b separated, and FIG. 24C showing them joined with a maneuvering gap between them.

FIG. 25 is an exploded perspective view of the two module 18a, 18b device with a removable microneedle protector 22, a skin adhesive layer 3, a rigid base 23, an optional dielectric base 24, compression pins 9, an improved separate cover 15, a microneedle array 4 with cantilevered arms 4b, a force touch sensor 7, all the above in module 18a and removable with an eject plastic sheet 25. This module sits above the reusable module with a lower rigid base 26 with an electronics unit (enclosed but not visible) inside a lower enclosure 11.

FIG. 26A is similar to FIG. 25, except that an extended cover 2 (but without a sidewall) is substituted for the separate cover 15. Note that an elastic dielectric base 24 is present to give greater flexibility and conformability to the microneedle array, for better contact and retention on the skin without movement. FIG. 26A is similar to FIG. 26B except that the latter has no elastic dielectric base.

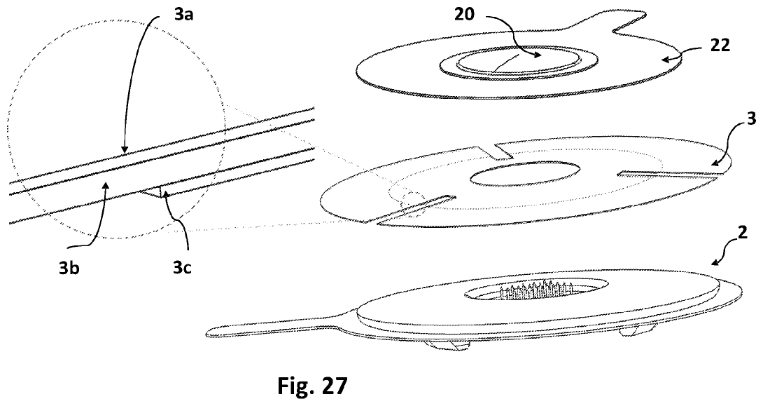

FIG. 27 is an exploded view of the reusable module 18a and a close up of one embodiment of the skin adhesive layer in three parts: 3a the skin facing adhesive, 3b a nonadhesive filament, and 3c adhesive to attach to the extended cover 2.

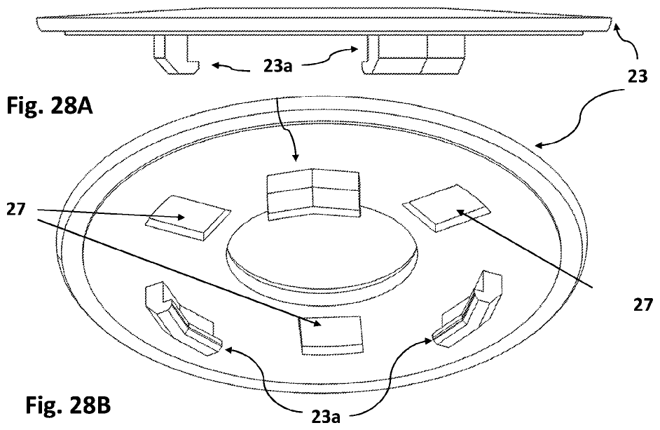

FIG. 28A is a side view of the rigid base 23 and FIG. 28B is a perspective view underneath, with the rigid base integrated with the elastic dielectric and the electrically conductive elastic e-contacts 9. The rigid base creates a robust connection between the disposable 18a and reusable electronic 18b modules of the invention with rigid hooks, which interlocks with the top of the electronics (i.e., the lower rigid base 26). [The provisional says so but is this right?]. Electric contacts 27 for the elastic conductive e-contacts 9 are also shown.

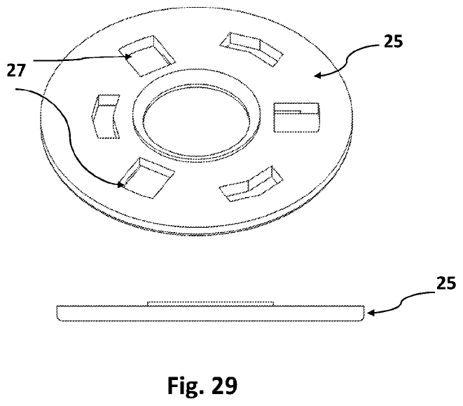

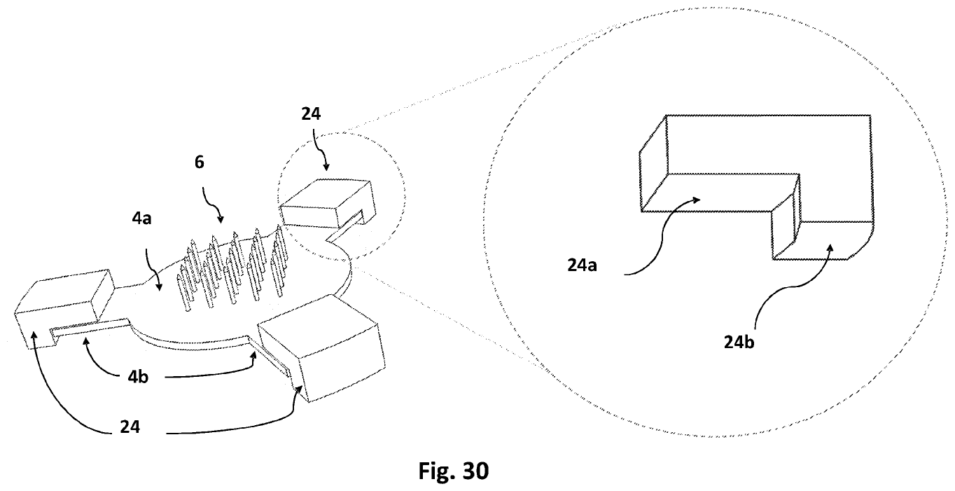

FIG. 29A is a side view and FIG. 29B is a perspective view of the elastic dielectric base 24, as also shown in FIG. 26A integrated between the rigid base (FIG. 28B) and the microneedle array (FIG. 30) creating insulation and sensor conformability to the skin. Due to its intrinsic elastic and hydrophobic materials properties, the elastic dielectric base 24 does the following: a) the provides sealing of the entire device against moisture, vapor, dust and liquids reaching electronics, and b) allows for reversible mechanical flexibility (spring-like reversibility) of the disposable/replaceable module 18a during wear. Additionally, the piece provides micro motion/shock absorbance (i.e., mechanical damping), reducing motion-based artifacts that would otherwise corrupt an acquired biosensor signal.

FIG. 30 is a perspective view of the microneedle array 4 with cantilevered arms and the compression pins 9a with an enlargement showing a contact region for the microneedle array 24a and for the electronics unit 24b. Elastic conductive e-contacts 9, shown in FIG. 30, are both conductive and elastic. Upon assembly of the disposable module 18a to the reusable electronics module 18a, one surface 24a of the elastic e-contact compresses against e-contact points on cantilevered arms 4b of the microneedle array and another surface 24b of the elastic e-contact presses against the electronic PCB contacts to create a reliable electrically conductive pathway from the microneedle tips to the PCB and vice versa. The electrically conductive layer and the electrical interconnections described in the '298 patent are incorporated in this pathway. This pathway is used for driving the biosensor and/or acquiring data from the biosensor by the electronics found within the rigid piece. Additionally, the materials properties and shape are elastically deformable minimizing the mechanical artifacts for the micro-shocks/vibrations (e.g., during running, exercising, etc.).

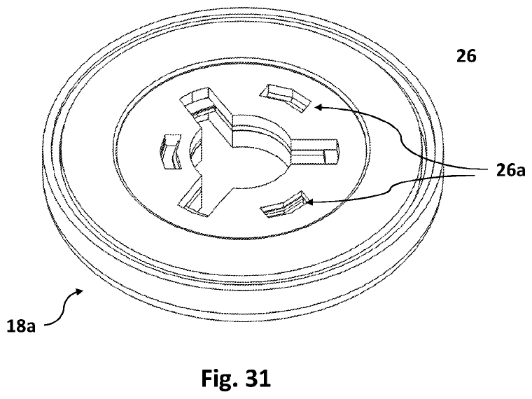

FIG. 31 is a perspective view of the reusable/electronics module 18a with the lower rigid base 26.

As described in the '298 patent, the extended cover and the improved separate cover 15 provide a) electrical isolation to the metalized surface of the microneedle array base, b) sealing/insolation against moisture (i.e., fluids and or gas vapors such as liquid or vaporized sweat, water etc.) created by filling of the microneedle cover vacant micro-interface by a curable material (i.e., photocurable or temperature curable resins, glue etc.), and c) added mechanical strength to the base of each individual microneedle.

The disposable module 18a is skin-conforming and in various embodiments can have four distinct mechanisms and characteristics which enable flexibility and conformability to skin:

Conformal skin-facing materials. The skin adhesive layer 3 of the disposable module is made from a flexible material with the proper (approximately 0.05-1 mm) thickness to allow for conformability. The surface area which makes contact with the skin is maximized in proportion to rigid material, resulting in a high flexible-to-rigid ratio for surface area, FIG. 2.

Degree of free motion. This is assisted by the elastic base contact to the rigid electronics body. An elastic non-conductive rubber dielectric base 24 forms the contact between the reusable electronic module 18a and the rigid base 23. By mechanically connecting the two pieces with an elastic material, the mechanical coupling between the two modules can designed to be loose.

Maneuvering gap. The maneuvering gap 28 mechanically decouples the rigid base 23 from the flexible piece. There is a small air gap between the bottom of the conformable skin adhesive and the top of the reusable electronic housing, which is created by the height of the rigid base and its clips. This space allows the rigid lower housing 11 to maneuver/tilt without making contact with the skin. This is done to mitigate the likelihood of mechanical coupling between the rigid electronic housing body and the skin. This aspect forms the “floating piece” design.

Counterbalance/cantilever microneedle array arms. The microneedle array 4 in some embodiments has cantilevered arms 4b which protrude outwards from the center of the array. Three arms depicted but in other embodiments there can be one or more arms). These cantilevered arms 4b provide a) electrical contact points for the elastic conductive rubber contacts (that sit on the top side of the array-piece), and b. provide mechanical contact with the printed circuit board (which is located beneath the array-piece). Directly underneath the microneedles themselves is a cavity. The arms, however, sit atop an electronic unit. In the integrated device, when a downward force is applied to the microneedles (e.g., from skin being pushed onto the microneedles), the flexible arms are loaded against the electronics unit which in turn causes an upward force to mechanically stabilize the microneedle array.

There are several structural features for microneedle/skin motion artifact interference via dynamic coupling through three distinct mechanisms. The integration and stacking of the disposable piece multilayers create a) flexibility and conformability of the disposable piece, b) insulation against the water, moisture, vapor, dust, etc., c) single step disposability of the entire disposable piece, and d) mitigated motion artifacts. Following is a description of each subcomponent and the structural features as well as the materials properties which support the mechanisms mentioned above.

First, the sloping skin-side of the upper surface 2b of the extended cover provides a constant skin insertion force while adherence to the skin (constant pushback of the skin/a natural spring on the microneedle array). When the wearable device is first attached to the skin using the adhesive, the skin curves with the surface of the domed surface. Due to the elastic properties of the skin, it tries to pull itself back to the body to become flat again. This pulling action is seen towards the edges of the domed surface, with larger pulling forces being caused by greater surface angle, a. This pulling from the outer edges causes a net pushing force of the skin found at the center of the dome toward the microneedle array, thus facilitating robust skin-microneedle contact, as shown in FIG. 2, a skin-facing rigid base curving away from the skin along its perimeter at angle α.

Next, cantilevered arms 4b of the microneedle array provide the second level of spring-like push-back force from the microneedle base towards the skin that enhances the coupling of the microneedle and skin at the microneedle/skin interface. Accordingly, the mechanical fluctuations/micro-movements caused by the wearer's physical motion are compensated for by the microneedle spring-like base where the elastic stress caused by the pinched skin loads the microneedle cantilevered arms and produces a reaction by the microneedle spring force back to the microneedle and keeps the two being in constant coupling interaction during the wearing of the sensor. Said differently, the process for these forces is as follows: Adhesive keeps the two pieces of skin and microneedle together and the skin depression at the well combines with the domed/curved surface of the rigid piece to create a bump on the skin with a net compression force towards the microneedle which loads the spring arms of the microneedles (strain gauge on the back) and creates a positive net force from the base of the microneedles to the skin. All of this creates the 3rd level mechanism of skin/microneedle coupling. Here, any force incident upon the microneedle array (which has no backing behind it), will load or strain the spring arms, which are backed by ledges found on the electronic PCB. This loading will produce an upward force of the array onto (into) the skin.

FIGS. 4, 5 and 6 among others show the microneedle array with the cantilevered arms 4b and the contact points to the PCB illustrated, allowing mechanical coupling of the microneedle/kin due to the compression positive force at the microneedle/skin interface caused by the cantilever arms

The lower rigid base 26, depicted in FIG. 31, mechanically and electrically interfacing with the disposable module 18a, comprises electronic hardware, such as a PCB assembled with components, which is used to: a) acquire data from the biosensor (located on the disposable module 18a), b) to sense mechanical interferences sources which cause artifacts upon the biosensor data, and c) apply artifact removal algorithms to cleanse the biosensor data of artifacts. An example of a mechanical interference sources is a (quasi-) static force applied to the integrated device thereby pushing the microneedles toward the skin. FIG. 31 shows the lower rigid base 26 with connection sites 26a to the disposable piece, namely the rigid hooks, a cavity for the microneedle array and cover to sit into and contact pads which provide an electrical connection between electronic hardware and the functionalized microneedle array via the conductive e-pads.

The reusable module 18a has mechanical features such as a maneuvering gap 28, as shown in FIG. 20C, which allows a tunable degree of freedom for the disposable component to flex and conform at to the skin of the wearer. It also has a waterproof, closed structure. The entire electronics system is water resistant by means of welding (e.g., ultrasonic), use of the photo-, or temperature-curable fluidic resins, or by the use of a double-sided adhesive and/or gluing the upper and lower enclosure piece together. Similarly, all the interfaces involving the PCB/enclosure contact are sealed using mentioned methods. FIG. 20C shows assembled pieces of the disposable module 18 including the array and reusable module electronics with sealed interfaces.

Another aspect of the cantilevered arms 4b is enabling a method to sense mechanical interference sources is a force touch sensor 7, for example a strain gauge, as described herein and shown for example in FIG. 7. A strain gauge (pressure sensor, or a touch sensor) on the back of the microneedle array and all of its cantilevered arms 4b. Using this configuration, the invention can electronically monitor the strain placed on the cantilever arms to approximate the force incident upon the microneedle array from the skin. These measurements can be used to digitally remove artifacts caused by forces placed upon the microneedle array which are more static in nature, such as if the user rests on the integrated sensor while sleeping. The strain gauge here is mechanically coupled due to the compression positive force at the skin and microneedle caused by movement of the cantilever arms.

Immobilization Anchoring is enabled by the invention. As a critical component for long term stability of the microneedle functional groups, the surface groups on the sleeve, various embodiments, are modified to optimize the uniformity and thickness of the first immobilization polymer layer. The surface groups therein act as anchors and create specific and non-specific bonding interactions with the immobilized layers. This anchoring also enhances the robustness of sensor and avoids delamination of the layers.

The surface of the cover can be highly manipulated to serve desired outcomes by incorporating materials distinct from the microneedle materials, thus having distinct properties. A schematic of this aspect is shown in FIG. 15. In different embodiments surface hydrophobicity of the sleeve can be adjusted to optimize the uniformity and thickness of the immobilized layers. Foam-like, spongy and porous structure allowing loading of materials such as water for skin hydration, pharmaceuticals or other compounds that impact the sensing mechanism constructively. To aid in protecting or concealing the microneedles, for example, foam-like squeezable long sleeves 5 can surround the microneedles. Additionally, these foam-like squeezable long sleeves and bodily features 8 can be loaded with drugs or other compounds for many purposes including improved multiplexed sensors (e.g., glucose and lactate), because the diffusion of hydrogen peroxide as by-products between the sensors highly diminishes the accuracy of the measurements. Reagents are loaded in or on the sleeves which decompose the produced hydrogen peroxide and thus avoid their diffusion between different sensors. One example of hydrogen peroxide decomposing reagents decomposing hydrogen peroxide is the enzyme catalase which produces oxygen and ameliorates the common issue of oxygen deficit in oxidase enzyme-based sensors.

In one embodiment the sleeve of the microneedle comprises a polymer comprising cavities containing solutions such as buffers to keep the microneedle sensing layers swollen and hydrated throughout their shelf life. This increases the stability and lifetime of the sensors. The already hydrated polymer layers on the microneedle sensors reduce warmup times upon their application to the body.

Sleeves greatly improve the mechanical robustness of microneedles due to the bigger stem diameter where sleeve covers the microneedle.

Anchoring from the cover body to the skin reduces noise artifacts caused by wearer's movements and sensors' micromovements on the body. A wide range of anchoring shapes and design are provided in various embodiments.

Drug delivery and other materials may be loaded on the sleeves, the bodily features and the cover 2a, 15. In various embodiments, cavities may be created on the surface of the sleeve or on the body of the cover. These cavities are loaded with reagents that aid in minimizing the biofouling and therefore, to impart longer life span to the microneedle-based sensors. Some examples of anti-biofouling reagents are dexamethasone and nitric oxide-releasing chemistry. Upon piercing the skin, microneedles are in contact with the interstitial fluid in the epidermis. Some small flow of interstitial fluid can occur toward the skin and extending to the microneedle base which causes the formation of a thin layer of the fluid being in contact with the cover. Thus, anti-biofouling reagent can diffuse to the fluid and the concentration gradient can cause it to diffuse toward the skin's inner layers. In FIG. 18, the bodily features 20 are spongy and loaded with a solution containing compounds for many purposes. The spongy features depress fully to allow the microneedles to pierce the skin.

Gradual release through enteric coating. The anti-biofouling reagents loaded cavities in some embodiments are coated with biocompatible polymer enteric coatings. Upon contact with the interstitial fluid, the enteric coating gradually dissolves which causes the gradual release of the reagents and their diffusion into the epidermis.

The improved cover as a distinct solid body from the microneedle array is a source of multiple functional features mentioned below. For example, some embodiments have metallic and nonmetallic traces on the body with following examples of functionality.

The improved cover in some embodiments may be electronic for skin-based sensors, integration of modalities, and robust electrochemical sensing. After insertion, the microneedle cover lies against the skin. This allows for skin-based (in addition to ISF-based) sensing modalities and techniques to be integrated into the wearable device. Moreover, skin-based stimulation techniques can be leveraged to improve reliability of ISF-based biosensing.

The following describes fabrication of an electronic microneedle cover and its application for sensing and stimulation. The electronic microneedle cover can be fabricated using additive (e.g., sputtering and photolithography) and/or subtractive (e.g., chemical etching) to form electrically conductive patterns. The substrate for the conductive patterns can comprise a variety of materials with various mechanical properties, e.g., a polyimide for a flexible cover. The improved cover can be composed of multiple substrate and conductive layers, with each substrate and/or layer being made of a different material, such as a flexible polyimide bonded to a rigid plastic.

Masks may be used in conjunction with additive and/or subtractive fabrication methods to form patterns of the conductive layers. The conductive material exposed to the skin can be controlled to present different electrical or mechanical coupling to the skin. For example, an exposed circular gold-plated copper pad can touch the skin to form a direct-coupled electrode. In another embodiment a circular copper pad which is covered with a thin polyimide film forms an electrode that is capacitively coupled to the body.

Interfaces and Interconnections. Multiple conductive layers can be interconnected with vias. Sections of the conductive layers can be exposed to allow for electrical connections to electronic hardware, e.g., a printed circuit board (PCB).

By using at least two electrodes (directly or capacitively coupled), the skin's impedance can be measured. Bioimpedance measurements are taken by applying an electric stimulation to the skin and measuring the skin's electrical response. The fundamental frequency of the stimulation can take various form which can be altered for different monitoring techniques-DC up to 1 MHz. An example of this is monitoring electrodermal activity (EDA), by which a DC or low frequency signal is applied to the skin to measure electrical characteristics of the skin (e.g., changes in skin conductance).

The conductive material can be patterned to form a temperature sensor. The conductive material of this sensor is electrically insulated from the skin. This forms a skin temperature monitor. Temperature data can be used in conjunction with amperometric biosensor data to compensate for drift.

Multiple sensing modalities can be used in conjunction with chemical biosensing to identify and capture physiological state. For example, EDA and skin-temperature monitoring, provided by the electronic microneedle cover, can be combined with cortisol sensing from the microneedles to identify states of stress for the device wearer.

Additional electrochemical sensors can be functionalized with electrodes on the microneedle cover.

In another embodiment a conductive trace may be used as a stimulator in order to reduce microneedle biofouling, thus improving reliability of microneedle biosensor measurements.

In one embodiment the conductive material on the microneedle cover is formed to create a heat producing element when an electrical signal (e.g., current) passes through it. This heat can be used to raise the local temperature of the skin. The effect of heating the skin is, without limitation, locally increased sweating, and blood or ISF flow, toward the skin.

The materials and shapes of the improved cover can be leveraged and designed to improve assembly repeatability (yields), increase skin insertion reliability, and improve the mechanical robustness of the microneedles. Reciprocal structures and guide holes can be used to reliably assemble the microneedle cover and the microneedles together.

The sleeves improve mechanical robustness to normal stress. Mechanical forces are thereby placed on materials which are better dealing with stress, offloading the forces from the microneedle itself.

To increase the reliability of insertion of the microneedles, the improved cover can be configured to produce spring-like controlled pushing out of the skin

Mechanical protrusions from walls surrounding each microneedle can be anchored into the skin (upon device application) to reliably insert the microneedles into ISF.

Methods for bonding the extended cover and the microneedle array include (1) fluid-based (e.g., running a fluidic material through the gap between the extended cover and the microneedle array and using curing methods such as UV and/or temperature to form chemical bonding) (2) glue/gel/solid-based, and (3) fusing or welding through e.g. ultrasound welding or high local heat to cause melting of the pieces together.

We claim:

1. A wearable biosensor device comprising
a. an extended cover comprising an electrically nonconductive material and facing a user's skin, and further comprising an upper surface integral with a lower surface comprising a set of first openings, the upper surface and the lower surface being joined by a slope,
b. a microneedle array comprising a substrate integral with microneedles, the microneedle array also comprising the electrically nonconductive material, and each of the microneedles comprising a tip, a body region and a base at the substrate, and the microneedles placed in complimentary position to the set of first openings in the extended cover, so that at least the tip of each of the microneedles rises beyond the lower surface of the extended cover, and an electrically conductive layer covers the microneedles and at least a portion of the substrate,
c. at least one chemical layer on the electrically conductive layer on at least one of the microneedles functionalized as a working electrode to interact with at least one target analyte in a biofluid beneath the user's skin and to produce electrical signals associated with analyte reactions detectable at the working electrode, and
d. an electronics unit positioned underneath the microneedle array connected electrically to the electrically conductive layer on the microneedles by a plurality of electrical interconnections.

2. The wearable biosensor device of claim 1 wherein the extended cover further comprises a fillet at the inner boundary of the upper surface to pinch the user's skin, to stretch the user's skin where the microneedles are inserted into the skin and to secure the microneedles at an insertion location of the user's skin.

3. The wearable biosensor device of claim 2 wherein the upper surface of the extended cover is higher at the fillet than at the outer edge of the extended cover.

4. The wearable biosensor device of claim 1 wherein the slope between the upper surface and the inner surface of the extended cover comprises one or several gradients.

5. The wearable biosensor device of claim 1 wherein the electrically nonconductive material is selected from the group consisting of nonconductive polymer, composites, ceramics and the like.

6. The wearable biosensor device of claim 1 wherein the extended cover further comprises sleeves integral with the lower surface positioned around at least a portion of the first openings, and there is a gap between the inner diameter of each of the sleeves and the outer diameter of one of the microneedles, a cured nonconductive polymer filling the gap and surrounding the base of the microneedle as a base structure to seal the gaps, strengthen the microneedle array, and form passivation of the microneedles leaving reproducible surface area of the electrically conductive layer on the microneedles.

7. The device as in claim 6 wherein the each of the sleeves has a width, a height and an angle which may be adjusted to modify insertion of the microneedles into the user's skin.

8. The device as in claim 1 wherein the lower surface of the extended cover further comprises at least one bodily feature extending toward the user's skin.

9. The device as in claim 8 wherein the at least one bodily feature comprises an absorbable, elastically deforming and spongy material.

10. The device as in claim 9 wherein the at least one bodily feature is impregnated with a fluid which may be released when pressure is applied.

11. The wearable biosensor device of claim 1, wherein the electronics unit comprises a data processing unit in communication with a signal processing circuit, the data processing unit comprising a processor and a memory and configured to process the electrical signal as data representative of one or more parameters of the analytes.

12. The wearable biosensor device of claim 11, wherein the signal processing circuit is configured to process the electrical signals by one or more of operations selected from the group consisting of amplifying the electrical signals, filtering the electrical signals, converting the electrical signals from analog to digital or modifying the electrical signals with non-analyte information, and wherein the data processing unit is configured to process the electrical signals after processing by the signal processing circuit.

13. The device as in claim 1 wherein the lower surface of the extended cover further comprises at least one conductive trace.

14. The device as in claim 13 wherein the conductive trace is configured as a non-analyte sensor to sense non-analyte conditions selected from the group consisting of perspiration sensor, force touch sensor, heat sensor and shock sensor, and further configured to send the non-analyte information as an electrical parameter to the signal processing circuit of the electronics unit for modification of the electrical signals associated with analyte reactions detectable at the working electrode.

15. The device as in claim 13 wherein the conductive trace is configured as an additional analyte sensor, and is further configured to send the additional analyte information as an electrical parameter to the signal processing circuit of the electronics unit for modification of the electrical signals associated with analyte reactions detectable at the working electrode.

16. The device as in claim 13 wherein the conductive trace is configured to heat or electrically stimulate the user's skin at the insertion location.

17. The wearable biosensor device as in claim 14 wherein the force touch sensor is positioned between the microneedle array and the electronics unit, and is selected from the group consisting of pressure, strain gauge, piezoelectric, piezoresistive, resonant, electromagnetic, capacitive, and diaphragm-based MEMS sensors operating individually or in combination with each other, and the force touch sensor is configured to transduce an applied force into an electrical parameter selected from the group consisting of resistance, current, capacitance, inductance, frequency or phase shift, voltage variability, optical or thermal changes and magnetic field variations.

18. The wearable biosensor device of claim 1 wherein the plurality of electrical interconnections comprise conductive e-contacts being either flexible or nonflexible, frictionous or compression-based.

19. The wearable biosensor device of claim 18 wherein the microneedle array further comprises a central portion where the microneedles are positioned, the central portion being integral to cantilevered arms secured to the conductive e-contacts, thus increasing potential movement of the central portion.

20. The wearable biosensor device of claim 1 comprising a replaceable module comprising the extended cover and the microneedle array and a reusable module comprising the electronics unit.

21. The wearable biosensor device of claim 20 wherein the replaceable module further comprises a dielectric base being either flexible or nonflexible.

22. The wearable biosensor device in claim 1 further comprises a skin adhesive layer on the upper surface of the extended cover configured to secure the wearable biosensor device and the microneedles to the user's skin at the insertion location.

23. The wearable biosensor device as in claim 1 wherein the microneedles are arranged into two or more sensing regions, wherein each of the sensing regions is configured as at least a two-electrode electrochemical system and further configured to be separated electrically from the other sensing regions, each of the sensing regions comprising at least one of the working electrodes functionalized with a portion of the at least one chemical layer configured to interact with a different one of the analytes, so that each of the sensing regions is configured to produce the electrical signals associated with the analyte reactions detectable at the working electrode.

24. The wearable biosensor device of claim 23 wherein each of the sensing regions further comprises at least one reference electrode and at least one counter electrode.

25. The wearable biosensor device of claim 1 wherein an under side of the lower surface of the extended cover and/or the substrate of the microneedle array further comprise microfluidic channels containing a cured custom resin.

26. The device of claim 5 wherein the extended cover comprises at least one second opening configured to feed a custom curable resin to fill the gaps between the sleeves and the microneedles and to create the base structures around the bases of the microneedles.

27. The wearable biosensor device of claim 1 wherein the plurality of electrical interconnections comprise
a. a plurality of electronic-connection holes in the substrate comprising at least one wall onto which the electrically conductive layer extends, and
b. conductive pins comprising a first and second end, wherein the first end of each of the conductive pins is connected to the electronic-connection holes and the second end of each of the conductive pins is connected to the electronics unit.

28. The wearable biosensor device of claim 27 wherein the electrically conductive layer is mechanically frictionous and the first end of each of the conductive pins is connected to the electronic-connection holes by a friction fit.

29. The wearable biosensor device of claim 1, wherein the analytes are selected from the group consisting of a metabolite, electrolyte, protein, amino acid, hormone, steroid, amine, nucleic acid, lipid, liposome, nanoparticle, and drug.

30. The wearable biosensor device of claim 1, wherein the chemical layer comprises a material which is selected from the group consisting of an enzyme, an ionophore, an antibody, a peptide nucleic acid (PNA), a DNA aptamer, a RNA aptamer, a molecularly imprinted polymer (MIP), and a cell.

31. The wearable biosensor device of claim 1, wherein the biofluid is selected from the group consisting of an interstitial fluid, an extracellular fluid, a cerebrospinal fluid, and blood.
